(19) United States
(12) Patent Application Publication
(10) Pub. No.: US 2013/0190607 A1
(43) Pub. Date: Jul. 25, 2013

(54) LOCAL COIL AND MANUFACTURE OF MECHANICALLY PREFORMED, FLEXIBLE LOCAL COILS FOR MRI

(71) Applicants: Stephan Biber, Erlangen/Frauenaurach (DE); Hubertus Fischer, Bamberg (DE); Thomas Kundner, Buckenhof (DE); Jörg Rothard, Litzendorf (DE)

(72) Inventors: Stephan Biber, Erlangen/Frauenaurach (DE); Hubertus Fischer, Bamberg (DE); Thomas Kundner, Buckenhof (DE); Jörg Rothard, Litzendorf (DE)

(73) Assignee: Siemens Aktiengesellschaft, Munchen (DE)

(21) Appl. No.: 13/554,901

(22) Filed: Jul. 20, 2012

(30) Foreign Application Priority Data
Jul. 21, 2011 (DE) 10 2011 079 571.5
Jul. 28, 2011 (DE) 10 2011 080 046.8

(51) Int. Cl. A61B 5/055; H01F 41/04

(52) U.S. Cl. CPC A61B 5/055; H01F 41/04
USPC 600/422; 29/605

(57) ABSTRACT

A method for manufacturing a local coil for a magnetic resonance tomography device includes manufacturing the local coil with a flat cross-section. The method also includes deforming the local coil from the flat cross-section into a curved cross-section.

This application claims the benefit of DE 10 2011 079 571.5, filed on Jul. 21, 2011. This application also claims the benefit of DE 10 2011 080 046.8, filed on Jul. 28, 2011.

### BACKGROUND

The present embodiments relate to a method for manufacturing a local coil for an MRT system and a local coil.

Magnetic resonance tomography devices for examining objects or patients using magnetic resonance tomography (MRT, MRI) are described, for example, in DE10314215B4.

In MR tomography, images with a high signal-to-noise ratio (SNR) may be recorded using local coils (e.g., coils, local coils). These are antenna systems that are attached in the direct vicinity of (anterior) or below (posterior) a patient. During the MRT measurement, the excited atoms induce a voltage into individual antennas of the local coil. The induced voltage is amplified with a low noise preamplifier (e.g., LNA, Preamp) and is routed via cables to the receiving electronics. High field systems (e.g., 1.5T to 12T and more) are also used in high resolution images to improve the signal-to-noise ratio.

Of importance in many clinical MR applications is the SNR of an image. SNR is effected by the local coil (e.g., antenna with active amplifiers), for example, by the losses in the antenna elements. Very small antennas enable a very high SNR in the vicinity of the antenna. On these grounds and on account of the possibility of an accelerated measurement by k-space sub-scanning (e.g., parallel imaging, SENSE, GRAPPA), there is interest in high channel (e.g., having many channels/antennas), very tight antenna arrays, the individual elements (e.g., antenna) of which may have a completely different alignment relative to the transmit field. Aside from the SNR, the simple applicability of the local coil is also an important property of the component, since during the time used to apply and position the local coil, the MRT system cannot be used for other purposes. A favorable arrangement of local coil elements together with a workflow-optimized mechanical design is important for the simultaneous optimization of SNR and workflow. Local coils exist in many designs, often dedicated to specific body regions (e.g., head, heart, prostate, knee, ankle, shoulder joint). Mechanically flexible local coils are known. The advantage of these flexible local coils lies in a relatively optimal molding on the body and the good SNR on account of the proximity of the local coil relative to the patient. For example, the variability of the patient anatomies in the abdomen/thorax region is very high and a flexible molding of the coil in at least one direction is advantageous.

Known flexible coils may be manufactured from mechanically flexible antenna supports that are incorporated into flexible foam materials. Additional regions for electronics are embodied to be rigid, but may possibly be so small that space remains therebetween for a bending of the antenna. In a normal shape, the local coils may be flat, for example, and only molded to the patient (and applied to his/her surface) by the application of belts that are fastened to the patient couch (e.g., by bending along the x-axis about the z-axis).

The dead weight of the local coil and the electronic housing contained in the local housing may be used to a certain degree for molding the local coil to the surface of the patient.

The coil may also be manufactured in a 3-dimensionally curved form.

### SUMMARY AND DESCRIPTION

The present embodiments may obviate one or more of the drawbacks or limitations in the related art. For example, a manufacturing method for a local coil for a magnetic resonance tomography device and/or a local coil may be further efficiently optimized.

An advantageous embodiment lies in a two-step method having a, for example, thermoplastic deformation of a flat local coil for improved molding of flexible MR local coils to patient anatomies.

A manufacturing method is, for example, provided that allows manufacture of a mechanically flexible coil having a “deformation.” “Deformation” may be, for example, that the coil is not flat in an initial state, but already exhibits a shape (e.g., slightly curved) to fit the anatomy of the patient that, during use, results in the coil resting in an improved fashion on the patient. This increases the SNR, and the coil is more easily fixable to the patient, since the deformation prevents or reduces a slipping of the coil when the coil is positioned on the patient and as a result, may even render the attachment of belts unnecessary.

A two-stage manufacturing method is proposed, for example, that includes the (a) manufacture of a flat local coil by, for example, conventional foam techniques. The method may also include (b) a thermoplastic deformation of the coil in order to achieve a desired “deformation” (e.g., a curved deformation).

Advantages of such a method (e.g., compared with a 3D deformed manufacture) may be, for example: 1. The molding costs may be minimal (e.g., for an alternative 3D deformation, possibly more complex tools may be required in a process step that may be a reason as to why this curved embodiment was still not considered); 2. Good manufacturing accuracy may be preserved, where simple positioning/fixing of one or more antennas in the local coil between the foam layers is possible during the thermal flat deformation process; and 3. Conventional flat coils and also coils molded in a curved state in accordance with the present embodiments may be manufactured using the same machines with the same outer geometry in accordance with manufacturing act a (by omitting act b for flat coils).

According to one embodiment, the deformed coil (e.g., the flat coil) may additionally be configured by elastic plastic parts that are incorporated in the foam (e.g., rods, surfaces, rubbers, springs) such that the deformation remains effectively in the desired form in the long term (e.g., in order to avoid that the plastic post forming in act b of the method is not adequately stable over the long term and the coil returning to an original flat shape by a creeping movement).

### BRIEF DESCRIPTION OF THE DRAWINGS

FIG. 1 shows a cross-section of one embodiment of a local coil fastened using belts to the chest of a patient on an MRT patient couch, to the left for a large patient and to the right for a small patient;

FIG. 2 shows a perspective view of one embodiment of a manufactured local coil;

FIG. 3 shows a top view of one embodiment of a manufactured local coil;

FIG. 4 shows a cross-section of one embodiment of a manufactured local coil;

FIG. 5 shows a flowchart of one embodiment of the manufacture of a local coil; and

FIG. 6 shows a schematic and simplified representation of one embodiment of an MRT system and a flat local coil.

### DETAILED DESCRIPTION OF THE DRAWINGS

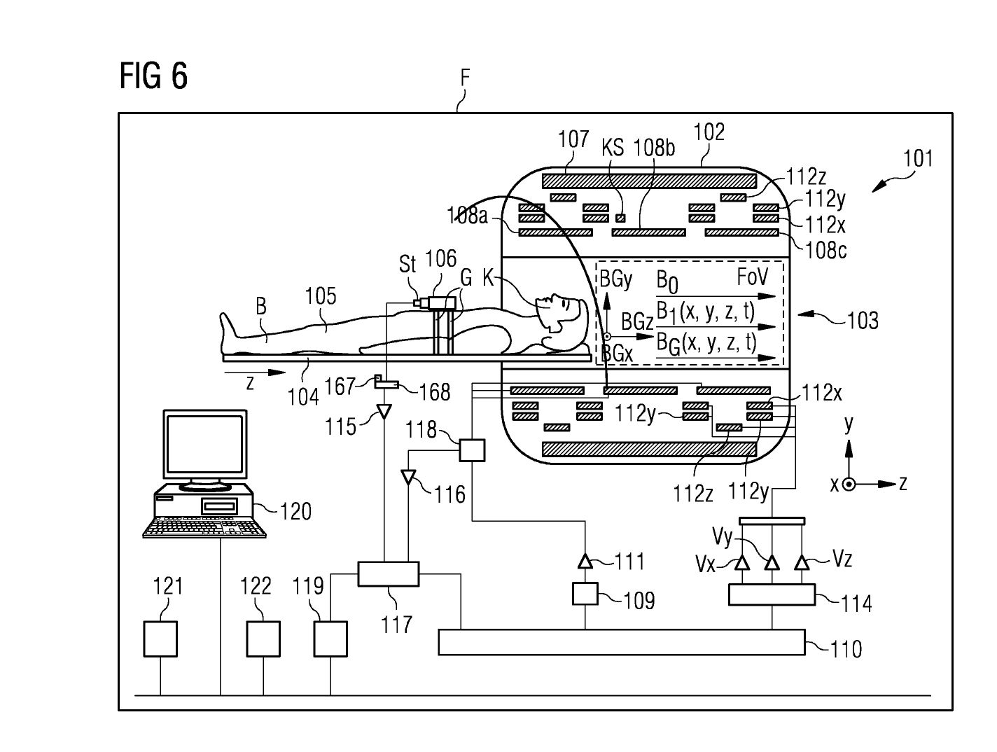

FIG. 6 shows an imaging magnetic resonance device MRT 101 (e.g., located in a shielded space or Faraday cage F) having a whole body coil 102 with a tubular space 103, for example, into which a patient couch 104 with a body 105 (e.g., an examination object or a patient) may be moved in the direction of arrow z (e.g., with or without local coil arrangement 106) in order to generate recordings of the patient 105 using an imaging method. A local coil arrangement 106 (e.g., fastened with the same or a further belt) is arranged on the patient (e.g., fastened with the belt), with which local coil arrangement 106 in a local region (e.g., field of view (FOV)) of the MRT, recordings of a subarea of the body 105 may be generated in the FOV. Signals of the local coil arrangement 106 may be evaluated (e.g., converted into images, stored or displayed) by an evaluation device (e.g., including elements 168, 115, 117, 119, 120, 121) of the MRT 101 that may be connected to the local coil arrangement 106 by way of a, for example, coaxial cable or by radio (e.g., element 167).

In order to examine the body 105 (e.g., an examination object or a patient) using a magnetic resonance device MRT 101 using magnetic resonance imaging, different magnetic fields that are attuned to one another as precisely as possible in terms of temporal and spatial characteristics are irradiated onto the body 105. A strong magnet (e.g., a cryomagnet 107) in a measuring cabin with a tunnel-type opening 103, for example, generates a strong static main magnetic field B₀, which amounts, for example, to 0.2 Tesla to 3 Tesla or more. A body 105 to be examined that is mounted on a patient couch 104 is moved into an approximately homogenous region of the main magnetic field B0 when viewed in the FOV. Excitation of the nuclear spin of atomic nuclei of the body 105 takes place by way of high frequency magnetic excitation pulses B1(x, y, z, t) that are irradiated by way of a high frequency antenna (and/or if necessary, a local coil arrangement), which is shown in FIG. 6 in very simplified form as a body coil 108 (e.g., a multipart body coil 108a, 108b, 108c). High frequency excitation pulses are generated, for example, by a pulse generation unit 109 that is controlled by a pulse sequence control unit 110. After amplification by a high frequency amplifier 111, the high frequency excitation pulses are routed to the high frequency antenna 108. The high frequency system shown in FIG. 6 is only indicated schematically. In other embodiments, more than one pulse generation unit 109, more than one high frequency amplifier 111 and several high frequency antennas 108 a, b, c are used in a magnetic resonance device 101.

The magnetic resonance device 101 has gradient coils 112x, 112 y, 112 z, with which magnetic gradient fields for selective layer excitation and local encoding of the measuring signal may be irradiated during a measurement. The gradient coils 112x, 112y, 112z are controlled by a gradient coil control unit 114 that, similarly to the pulse generation unit 109, is connected to the pulse sequence control unit 110.

Signals emitted by the excited nuclear spin (e.g., the atomic nuclei in the examination object) are received by the body coil 108 and/or at least one local coil arrangement 106, amplified by assigned high frequency preampflier 116 and further processed and digitized by a receive unit 117. The recorded measuring data is digitized and stored in a k-space matrix as complex numerical values. An associated MR image may be reconstructed from the k-space matrix populated with values using a multidimensional Fourier transformation.

For a coil that may be operated both in the transmit and also in the receive mode (e.g., the body coil 108 or a local coil 106), the correct signal forwarding is controlled by an upstream transmit-receive switch 118.

An imaging processing unit 119 generates an image from the measuring data, which is shown to a user by way of a console terminal 120 and/or is stored in a storage unit 121. A central computing unit 122 controls the individual system components.

In MR tomography, images with a high signal-to-noise ratio (SNR) may be recorded using local coil arrangements (e.g., coils, local coils). These are antenna systems that are attached in the direct vicinity of (anterior), below (posterior), on, or in the body 105. With an MR measurement, the excited nuclei induce a voltage into the individual antennas of the local coil. The induced voltage is amplified with a low noise preamplifier (e.g., LNA, Preamp) and is forwarded to the receiving electronics. In order to improve the signal-to-noise ratio in high resolution images, high field systems are used (e.g., 1.5T and more). If more individual antennas may be connected to an MR receiving system than there are receivers available, a switching matrix (e.g., RCCS) is integrated between the receiving antennas and the receiver, for example. This routes the currently active receiving channels (e.g., the channels that lie precisely in the FOV of the magnet) to the available receiver. As a result, more coil elements than there are receivers available may be connected, since with whole body coverage, only the coils that are in the FOV and/or in the homogeneity volume of the magnet are to be read out.

An antenna system may be referred to as a local coil arrangement 106, for example, that may include, for example, one antenna element or an array coil including several antenna elements (e.g., coil elements). The individual antenna elements are embodied, for example, as loop antennas (e.g., loops), butterfly, flexible coils or saddle coils. A local coil arrangement includes, for example, coil elements, a preamplifier, further electronics (e.g., shetah current filters), a housing, bearing surfaces and may include a cable with a plug, by which the local coil arrangement is connected to the MRT system. A receiver 168 attached on the system side filters and digitizes a signal received by a local coil 106 (e.g., by radio) and transfers the data to a digital signal processing facility. The digital signal processing facility may derive an image or a spectrum from the data obtained by a measurement and make the image or spectrum available to the user for subsequent diagnosis thereby or storage.

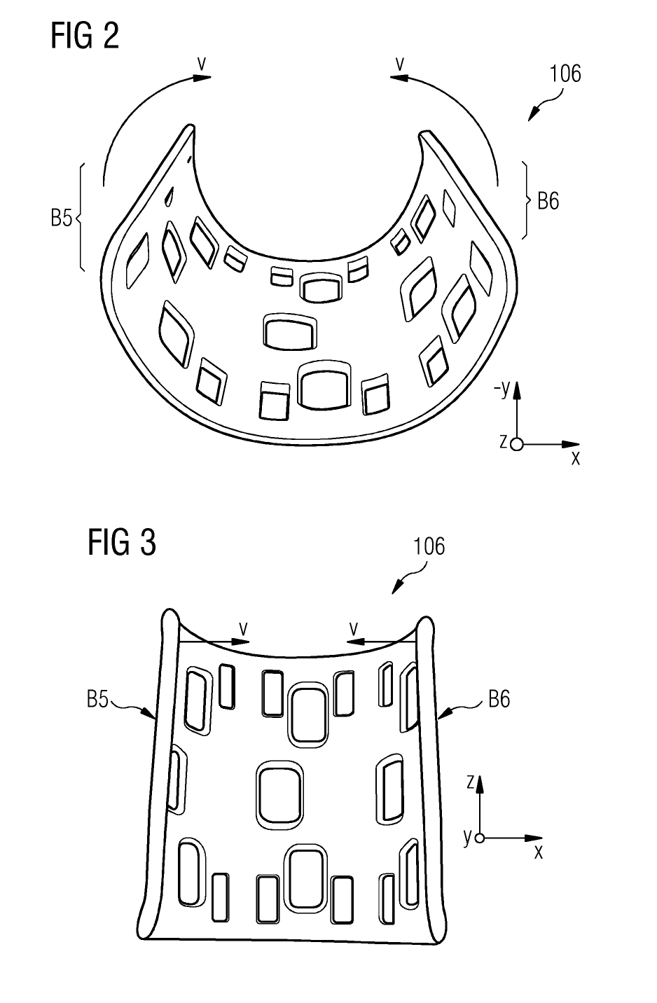

Exemplary embodiments of MRT local coils manufacturing methods and local coils are described in more detail with the aid of FIGS. 2-5.

A patient 105 lying on a patient couch 104 is to be examined in an MRT 101 using a magnetic resonance tomography device local coil 106 resting on his/her chest, abdomen or on another body part.

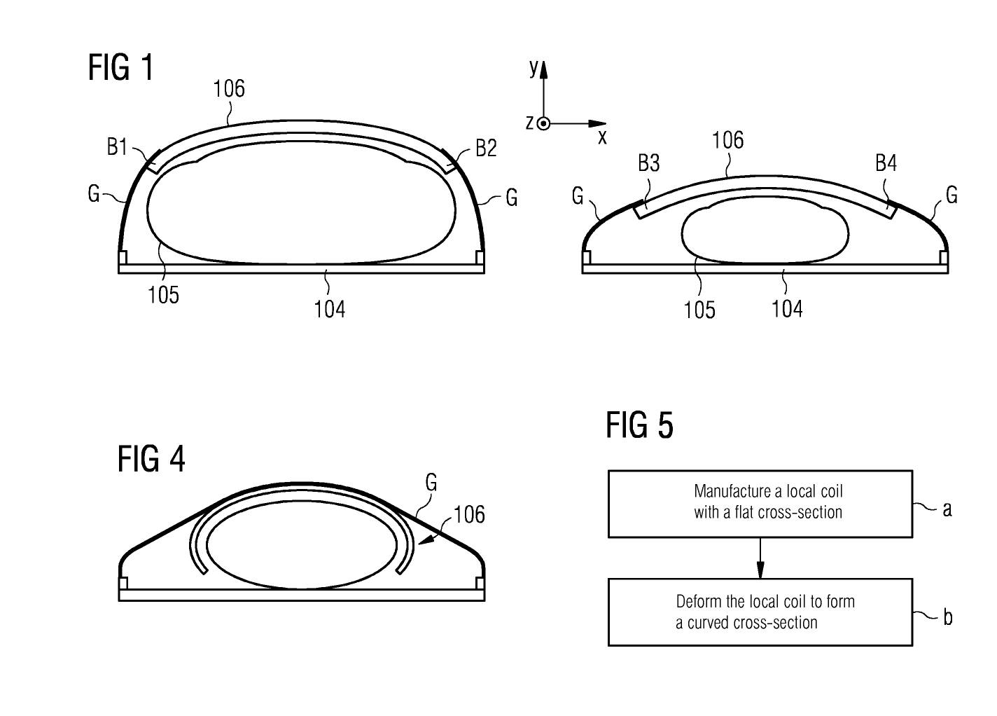

FIG. 1 shows a cross-sectional representation of a local coil 106 fastened to the chest of a patient 105 on an MRT patient couch 104 using one or several belts G.

The local coil 106, viewed in the cross-section (section in the plane x-y in FIG. 6 through the coil), for example, in an unbent initial state, is flat (e.g., as in FIG. 6, like a board) and may be elastically deformed all over or in the lateral regions by bending in directions v relating to the patient until a curved cross-section is reached to achieve close attachment of the local coil 106 with the surface of the patient 105 also in the lateral area (e.g., the flanks) of the local coil.

An elastic deformation of the local coil 106 from a flat cross-section, in the unbent state, to a curved cross-section shown in FIG. 1 (e.g., for close attachment of the local coil 106 on the surface of the patient 105) takes place, for example, by stretching belts G that engage with the local coil 106 and the patient couch 104 using belt tabs, for example.

In the example to the right in FIG. 1, this results in a suboptimal attachment of the local coil on thin (e.g., narrow in the x-direction) patients 105 in the lateral/flank region B3, B4 of the local coil 106 (e.g., protruding laterally without contact with the patient) compared with the relatively improved (closer) attachment of the local coil on fatter patients 105 in the lateral flank region B1, B2 of the local coil 106 to the left in FIG. 1 (e.g., resting on the patient).

FIG. 2 shows a perspective view, FIG. 3 shows a top view and FIG. 4 shows a cross-section of one embodiment of a manufactured local coil 106 with, in the unbent state of the local coil, a curved cross-section (e.g., for closer attachment of the local coil 106 to the surface of the patient 105 even in the lateral region). In this way, an elastic bending of the local coil 106 into a more flat (e.g., less curved) and/or into a more intensively curved state (e.g., than in the unbent initial state in FIG. 2-4) is also possible from this already unbent, curved cross-section of the local coil 106 according to FIGS. 2-4. On account of the arching present in the unbent initial state in FIGS. 2-4, compared with the flat initial state of a conventional local coil 106, attachment of the local coil in the lateral region B5, B6 of the patient may take place with little or no pulling (e.g., by belts) in the direction, for example, v relative to the patient. For many patients with approximately average anatomies, belts may not even be necessary.

FIG. 5 shows a flowchart simplifying two acts a, b of the manufacture of a local coil 106 for a magnetic resonance tomography device 101. In act a, the local coil 106 is manufactured with a flat cross-section (x-y plane) including, for example, a thermoplastic with or without elastic cores (e.g., similarly from a thermoplastic) such as rods and flat elements. In act b, the planar local coil 106 is, for example, thermally deformed into a curved cross-section (x-y plane) by, for example, heating while the local coil rests on a curved shape.

A manufacturing method is therefore provided that allows for manufacture of a mechanically flexible coil with a “deformation.”

In one embodiment of the method, the thus deformed coil may be configured by further elastically bendable/spring plastic parts that are incorporated in the foam (e.g., rods, surfaces, rubbers, springs) such that the deformation remains in the desired shape for a satisfactory period of time.

One advantage of a two-step method with a, for example, thermoplastic deformation of an existing local coil may lie in an efficient, good molding of flexible MR local coils to patient anatomies.

Coil electronics of the local coil may be integrated, for example, only after the second thermal deformation act b.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for manufacturing a local coil for a magnetic resonance tomography device, the method comprising:
manufacturing the local coil with a flat cross-section; and
deforming, as part of the manufacturing, the local coil with the flat cross-section into a curved cross-section.

2. The method as claimed in claim 1, wherein manufacturing comprises manufacturing the local coil with the flat cross-section with a cross-section having a section at least approximately at right angles to a provided longitudinal patient direction or head-foot direction of the local coil.

3. The method as claimed in claim 1, wherein manufacturing comprises manufacturing the local coil as mechanically flexible.

4. The method as claimed in claim 3, wherein manufacturing comprises manufacturing the local coil as elastically bendable at least about a longitudinal patient axis, the z-axis, or the longitudinal patient axis and the z-axis.

5. The method as claimed in claim 1, wherein manufacturing comprises using a foam technique.

6. The method as claimed in claim 1, wherein deforming comprises thermoplastically deforming the local coil into the curved cross-section.

7. The method as claimed in claim 1, wherein manufacturing the local coil comprises manufacturing a chest coil, a thorax coil, an abdomen coil, a head coil, a heart coil, a knee coil, an ankle coil or a shoulder joint coil.

8. The method as claimed in claim 1, wherein manufacturing comprises manufacturing the local coil such that the local coil has a shape adjusted to an average patient anatomy in an unbent initial state.

9. The method as claimed in claim 1, wherein manufacturing comprises manufacturing the local coil such that the local coil has a surface adjusted to a body surface of an average patient.

10. The method as claimed in claim 1, wherein manufacturing comprises manufacturing the local coil such that, in an unbent initial state, the local coil has a shape adjusted to an average patient anatomy.

11. The method as claimed in claim 1, wherein manufacturing comprises manufacturing the local coil such that, in an unbent initial state, the local coil has a surface adjusted to a body surface of an average patient.

12. The method as claimed in claim 1, wherein manufacturing comprises manufacturing the local coil such that the local coil is elastically bendable wholly or only in a region of a lateral flank, from the curved cross-section into a greater or lesser curved cross-section.

13. The method as claimed in claim 1, wherein manufacturing comprises incorporating spring plastic parts.

14. The method as claimed in claim 13, wherein the incorporating spring plastic parts comprises incorporating rods, flat elements, rubbers, springs, or a combination thereof.

15. The method as claimed in claim 1, wherein manufacturing comprises manufacturing the local coil with a flat longitudinal section, the flat longitudinal section being a section at least approximately vertical relative to an axis passing through both shoulders of a patient.

16. A local coil for a magnetic resonance tomography device, the local coil comprising:
a curved cross-section in a resting state, the curved cross-section incorporating a deformation from a flat cross-section; and
flexible material allowing bending of the curved cross-section in application to a patient.

17. The local coil as claimed in claim 16, wherein the flexible material is mechanically flexible.

18. The local coil as claimed in claim 17, wherein the flexible material is elastically bendable about a longitudinal axis of the patient, the z-axis, or the longitudinal axis of the patient and the z-axis.

19. The local coil as claimed in claim 16, wherein the local coil is manufactured from a thermoplastic at least partially, in a housing of the local coil, or at least partially and in the housing of the local coil.

20. The local coil as claimed in claim 16, wherein the local coil is a chest coil, a thorax coil, an abdomen coil, a head coil, a heart coil, a knee coil, an ankle coil or shoulder joint coil.

21. The local coil as claimed in claim 16, wherein in an unbent initial state, the local coil has a shape adjusted to an average patient anatomy.

22. The local coil as claimed in claim 21, wherein in the unbent initial state, the local coil has a surface adjusted to a body surface of an average patient.

23. The local coil as claimed in claim 16, wherein the local coil is curved in an unbent initial state.

24. The local coil as claimed in claim 16, wherein in an unbent initial state, the local coil has a shape adjusted to an average patient anatomy.

25. The local coil as claimed in claim 24, wherein in the unbent initial state, the local coil has a surface adjusted to a body surface of an average patient.

26. The local coil as claimed in claim 16, wherein the local coil is bendable wholly or only in a region of a lateral flank from the curved cross-section elastically into a greater or lesser curved cross-section.

27. The local coil as claimed in claim 16, further comprising spring plastic parts.

28. The local coil as claimed in claim 27, wherein the spring plastic parts comprise rods, flat elements, rubbers, springs, or a combination thereof.
